(19) United States
(12) Patent Application Publication
(10) Pub. No.: US 2002/0110843 A1
(43) Pub. Date: Aug. 15, 2002

(54) Compositions and methods for epitope mapping

(76) Inventor: David P. Dumas, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/853,457

(22) Filed: May 10, 2001

Related U.S. Application Data
(60) Provisional application No. 60/325,766, filed on May 12, 2000.

(51) Int. Cl. G01N 33/53; G01N 33/537; G01N 33/543; C12Q 1/68

(52) U.S. Cl. 435/7.92; 435/6

(57) ABSTRACT

The invention provides a composition comprising a diverse population of reagent ligands attached to a solid support and a diverse population of reagent antibodies specifically bound to the reagent ligands. The ligands can be peptides, oligosaccharides, oligonucleotides, or organic molecules. The invention additionally provides methods of determining an epitope in a sample contacting a composition comprising a diverse population of ligands attached to a solid support and a diverse population of antibodies specifically bound to each of the ligands with a sample; and detecting the antibodies bound to the diverse population of ligands. The invention further provides methods of diagnosing a disease, identifying a potential therapeutic agent, and mapping accessible epitopes of a polypeptide using invention compositions.

This application claims the benefit of priority of U.S. Provisional application Ser. No. ______, filed May 12, 2000, which was converted from U.S. Ser. No. 09/569,713, filed May 12, 2000, the entire contents of which is incorporated herein by reference.

### BACKGROUND OF THE INVENTION

The present invention relates generally to drug development and diagnostics and more specifically to immunological assays for determining epitope expression.

Greater than 300,000 different proteins are estimated to be present in humans. Of these proteins, there are about 15,000 potential molecular therapeutic targets. To date, less than 1000 have been identified and exploited for pharmaceuticals. In an attempt to identify which of the remaining 299,000 proteins are viable pharmacological targets, various genomic tools have been developed to analyze anomalies in the genetic code or mRNA levels.

Genomics has been developed over the last decade in part to identify new targets and has led to the development of new diagnostic methods. Leads identified by changes in mRNA levels have fueled the high throughput screening groups of the major pharmaceutical companies, many of which screen as many as 100 targets per year. The genomics approach is, however, limited in that a disease is manifested at the protein level. Therefore, the changes in mRNA levels that form the cornerstone of genomics is a poor approximation for biochemical changes in a diseased tissue. Biological function, or aberrant function, is the result of changes in protein levels or processing. The correlation between mRNA levels and protein expression is less than a 0.5 (Anderson and Seilhamer, Electrophoresis 18:533-537 (1997)). With the measurement of changes in mRNA using the tools of genomics, the actual biologically active species, the proteins, are not assessed. In addition, genomic analysis has no way of identifying changes in post translational modification, such as glycosylation or phosphorylation. It is only by direct analysis of the proteins that changes indicative of a disease will become evident.

Consequently, the actual success rate for genomic leads consequently is very low. Following identification of a lead from genomics analysis, the protein must be expressed in a variety of cell or animal models in order to attribute functionality or some correlative property between the protein and a disease. The direct measurement of protein levels or processing within a diseased tissue would greatly enhance the success rate of target identification and eliminate some of the intermediate steps necessary for validating a target.

In part due to the limitations of genomic analysis and in part due to the need to functionally characterize genomic leads, the field of proteomics was developed. In spite of its acknowledged advantages over genomics for identifying biologically significant changes in protein levels as the result of a disease state, proteomics has lagged in its incorporation into the biotechnology sector and drug discovery efforts. This shortcoming is the result of reliance on the adaptation of old techniques to proteomics studies, particularly mass spectroscopy and 2-D electrophoresis. While these techniques have been available for over thirty years, automation, reproducibility, quantification, and rapid throughput have proven to be formidable hurdles blocking the incorporation of proteomics into the discovery stream of biotechnology.

The traditional techniques for proteomics, 2D-electrophoresis and mass spectroscopy, are technically limiting in that only about 20% of the proteins loaded on a 2D-electrophoresis gel are visible, and of those, only the proteins with masses ranging between 10 kDa and 100 kDa are readily separated. Relevant expression differences are difficult to assign and validate since multiple gels are difficult to prepare in a reproducible manner. As a result of these technical hurdles, the study of proteomics has not found its place in the drug discovery pipeline.

Thus, there exists a need for convenient and efficient methods to analyze proteins and modifications thereof for drug discovery and diagnostic purposes. The present invention satisfies this need and provides related advantages as well.

### SUMMARY OF THE INVENTION

The invention provides a composition comprising a diverse population of reagent ligands attached to a solid support and a diverse population of antibodies specifically bound to the reagent ligands. The ligands can be peptides, oligosaccharides, oligonucleotides, or organic molecules. The invention additionally provides methods of determining an epitope in a sample by contacting a composition comprising a diverse population of reagent ligands attached to a solid support and a diverse population of antibodies specifically bound to the reagent ligands with a sample; and detecting the antibodies bound to the diverse population of reagent ligands. The invention further provides methods of diagnosing a disease, identifying a potential therapeutic agent, and mapping accessible epitopes of a polypeptide using invention compositions.

### BRIEF DESCRIPTION OF THE DRAWINGS

FIG. 1 shows an outline for determining epitope expression. In step A, a combinatorial peptide library is synthesized on a solid support. In step B, antibodies are specifically bound to the peptides to form a ProtoChip. In step C, a sample is applied to the ProtoChip. In step D, epitopes expressed in the sample competitively bind to the antibodies. In step E, antibodies remaining bound to the peptides are visualized.

FIG. 2 shows the construction of a peptide library.

FIG. 3 shows the ScFv plasmid for expression of a recombinant antibody library.

### DETAILED DESCRIPTION OF THE INVENTION

The present invention provides a composition comprising a plurality of reagent ligands attached to a solid support and a plurality of reagent antibodies specifically bound to the ligands, which is termed a ProtoChip. The ligands can be peptides, oligonucleotides, oligosaccharides or other organic molecules. The invention also provides methods of determining epitope expression in a sample using a ProtoChip. The present invention draws from the fields of molecular biology, immunology, combinatorial chemistry, and high throughput screening. The present invention can be advantageously used to overcome the difficulties associated with traditional proteomics techniques such as mass spectroscopy and 2-D electrophoresis.

The present invention provides an advancement of useful proteomics techniques that uses aspects of the competitive immunoassay and is readily automatable for the mapping of the epitome, an analysis of epitopes expressed in a cell. The present invention provides a method that is rapid, reproducible, quantifiable, and provides an accurate snapshot of the proteome. Among many applications, the present invention can be applied to drug target discovery, diagnostics, drug development, pharmacoproteomics, agricultural biotechnology, and structural bioinformatics.

The invention ProtoChip has advantages over current proteomics methodology. Essentially all possible epitopes can be quantified using the invention ProtoChip, with no size restriction for proteins or peptides. All proteins that can be solubilized, even membrane bound proteins that are difficult to analyze by traditional proteomics techniques such as 2D electrophoresis, can be quantified with the invention ProtoChip. The invention allows for highly reproducible results, which can be readily compared from experiment-to-experiment. The invention allows detection of proteins 2 to 3 orders of magnitude lower in concentration than by electrophoresis. Known proteins can be easily quantified using methods of the invention.

The invention can be used in diagnostic applications and provides advantages similar to those observed with nucleic acid based diagnostics. These advantages include product standardization, miniaturization, automation, and information management. The invention provides advantages over other immunochemistry based assays, including improved sensitivity and specificity and allowing simultaneous analysis of multiple epitopes. The invention is also advantageous in that automation of all steps of sample processing can be readily achieved.

As used herein, a “ligand” refers to a molecule that can specifically bind to an antibody. The term specifically means that the binding interaction is detectable over non-specific interactions by a quantifiable assay. A ligand can be essentially any type of molecule such as a peptide or polypeptide, nucleic acid or oligonucleotide, carbohydrate such as oligosaccharides, or any organic derived compound.

As used herein, a “reagent ligand” refers to a ligand used as a reagent for analysis of a sample, that is, a non-analyte ligand. Although a reagent ligand can be derived from a natural source or chemically synthesized, it is understood that a reagent ligand specifically excludes ligands in a sample to be analyzed. As used herein, the term reagent ligand specifically excludes antibodies, that is, the reagent ligand is a non-antibody ligand.

As used herein, the term “polypeptide” refers to a peptide, polypeptide or protein of two or more amino acids. A polypeptide can also be modified by naturally occurring modifications such as post-translational modifications or synthetic modifications, including phosphorylation, lipidation, prenylation, sulfation, hydroxylation, acetylation, addition of carbohydrate, addition of prosthetic groups or cofactors, formation of disulfide bonds, proteolysis, assembly into macromolecular complexes, and the like.

A modification of a peptide can also include non-naturally occurring derivatives, analogues and functional mimetics thereof generated by chemical synthesis. Derivatives can include chemical modifications of the polypeptide such as alkylation, acylation, carbamylation, iodination, or any modification that derivatizes the polypeptide. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine. Also included as derivatives or analogues are those polypeptides which contain one or more naturally occurring amino acid derivatives of the twenty standard amino acids, for example, 4-hydroxyproline, 5-hydroxylysine, 3-methylhistidine, homoserine, ornithine or carboxyglutamate, and can include amino acids that are not linked by peptide bonds.

As used herein, the term “nucleic acid” or “oligonucleotide” means a polynucleotide such as deoxyribonucleic acid (DNA) or ribonucleic acid (RNA). As used herein, the term “oligosaccharide” refers to polymers of monosaccharides that can be linear or branched. Oligosaccharides include modifications of monosaccharides. As used herein, the term “organic molecule” refers to organic molecules that are chemically synthesized or are natural products.

As used herein, the term “antibody” is used in its broadest sense to include polyclonal and monoclonal antibodies, as well as antigen binding fragments of such antibodies. An antibody useful in the invention, or antigen binding fragment of such an antibody, is characterized by having specific binding activity for a ligand or sample epitope of at least about 1×10⁵ M⁻¹. Thus, Fab, F(ab′)₂, Fd, Fv, single chain Fv (scfv) fragments of an antibody and the like, which retain specific binding activity for a ligand, are included within the definition of an antibody. Specific binding activity of an antibody for a ligand can be readily determined by one skilled in the art, for example, by comparing the binding activity of an antibody to a particular ligand versus a control ligand that differs from the particular ligand. Specific binding can similarly be determined for a binding molecule for the ligand that is not an antibody. Methods of preparing polyclonal or monoclonal antibodies are well known to those skilled in the art (see, for example, Harlow and Lane, Antibodies: A Laboratory Manual, Cold Spring Harbor Laboratory Press (1988)).

In addition, the term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains as described by Huse et al. (Science 246:1275-1281 (1989)). These and other methods of making functional antibodies are well known to those skilled in the art (Winter and Harris, Immunol. Today 14:243-246 (1993); Ward et al., Nature 341:544-546 (1989) Harlow and Lane, supra, 1988); Hilyard et al., Protein Engineering: A practical approach (IRL Press 1992); Borrabeck, Antibody Engineering, 2d ed. (Oxford University Press 1995)).

A particularly useful method for generating antibodies is based on using combinatorial libraries consisting of variable heavy chains and variable light chains (Kang et al., Proc. Natl. Acad. Sci. USA, 88:4363-4366 (1991), Huse et al., Science 246:1275-1281 (1989)). The advantage of using such a combinatorial antibody library is that antibodies do not have to be individually generated for each ligand of the ProtoChip. No prior knowledge of the exact characteristics of the ligands on the ProtoChip is required when using a combinatorial antibody library.

As used herein, a “reagent antibody” refers to an antibody used as a reagent for analysis of a sample, that is, a non-analyte antibody. Although a reagent antibody can be derived from a natural source, chemically synthesized, or expressed recombinantly, it is understood that a reagent antibody specifically excludes antibodies in a sample to be analyzed. Similarly, a “reagent binding molecule” such as a reagent receptor, polypeptide or enzyme, as disclosed herein, is a binding molecule used as a reagent for analysis of a sample, that is, a non-analyte binding molecule.

As used herein, the term “population” is intended to refer to a group of two or more different molecules. Populations can range from two to tens to hundreds to thousands, or even millions or billions or more molecules. For example, a population can contain about 3 or more, about 5 or more, about 7 or more, about 10 or more, about 15 or more, about 20 or more, about 30 or more, about 40 or more, about 50 or more, about 75 or more, about 100 or more, about 200 or more, about 500 or more, or even about 1000 or more molecules. A population can also contain about 10⁴ or more, about 10⁵ or more, about 10⁶ or more, about 10⁷ or more, about 10⁸ or more or about 10⁹ or more molecules, about 10¹⁰ or more molecules, about 10¹¹ or more molecules, about 10¹² or more molecules, or even greater numbers of molecules. As used herein, a “subset” when used in reference to a population refers to group of molecules that is less than all of the population.

As used herein, a molecule in a sample can be essentially any type of molecule such as a polypeptide, nucleic acid, carbohydrate, lipid, or any organic derived compound. Moreover, derivatives and analogues are also intended to be included within the definition of this term. For example, polypeptides can be modified by postranslational modifications or synthetic modifications, including phosphorylation, lipidation, prenylation, sulfation, hydroxylation, acetylation, addition of carbohydrate, addition of prosthetic groups or cofactors, formation of disulfide bonds, proteolysis, assembly into macromolecular complexes, and the like.

The invention provides a composition comprising a diverse population of reagent ligands attached to a solid support and a diverse population of antibodies specifically bound to the reagent ligands. Such a composition is also termed a ProtoChip. The ligands can be peptides, oligosaccharides, oligonucleotides, or organic molecules.

The present invention provides compositions and methods useful for determining the expressed epitopes of a molecule in a sample from an individual. The methods of the invention are particularly useful for mapping epitopes on polypeptides expressed in a sample. Epitope mapping has been described as a means to identify the specific site to which an antibody binds on the surface of a polypeptide. Traditionally, epitope mapping has been done by synthesizing all the 5 to 15 amino acid stretches of a known protein with a known sequence, where the peptides are offset from each other by 3 to 10 amino acids. The peptide epitope is identified as the one that complexes with the antibody.

The present invention provides methods allowing epitopes present and accessible on essentially any polypeptide or molecule in a sample to be determined. The invention is advantageous in that no prior knowledge of the sample polypeptide or sequence is required, and the analysis of samples containing unknown protein mixtures becomes feasible.

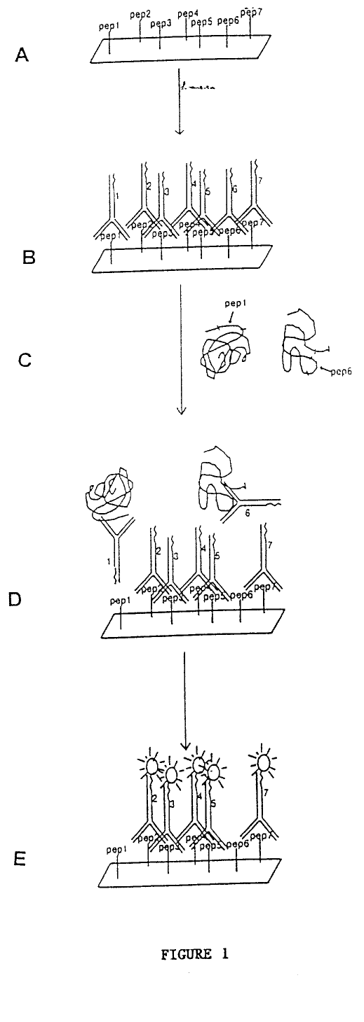

The invention provides a ProtoChip, which is a diverse population of reagent ligands attached to a solid support and a diverse population of reagent antibodies specifically bound to the ligands. In one embodiment, the ligands are peptides attached to a solid support and are essentially an immobilized combinatorial epitope peptide library made up of combinations of amino acids (FIG. 1, step A). The ligands, which have binding activity for antibodies, can be complexed with antibodies to form a ProtoChip (FIG. 1, step B). The antibodies can be, for example, antibodies expressed as recombinant ScFv.

The ProtoChip functions to detect the presence of epitopes in a sample. If a sample is exposed to a ProtoChip, those epitopes present in the sample and accessible to antibody binding compete for binding of antibodies to ligands (FIG. 1, steps C and D). Thus, antibodies having binding activity for epitopes present in the sample, for example, epitopes on the surface of polypeptides, are displaced from their specific ligand epitope when exposed to competing epitopes in the sample.

The invention thus also provides a composition comprising a diverse population of reagent ligands attached to a solid support and a diverse population of reagent antibodies specifically bound to a subset of the reagent ligands, wherein an unbound ligand has binding activity for an antibody having specificity for a molecule in a sample (FIG. 1).

The antibodies remaining bound to the subset of ligands can be detected (FIG. 1, step E). By identifying the ligands which are unbound by antibody, that is, ligands having binding activity for the displaced antibodies specific for a molecule in a sample, epitope expression in the sample can be determined. Thus, a map of epitopes is generated that provides a proteome fingerprint for a sample such as a biological fluid. The present invention provides methods that are accurate, reproducible, and fast. The methods can be applied to pharmaceutical target identification, drug discovery, diagnostics, pharmacoproteomics, structural bioinformatics, agricultural biotechnology, drug development, and species identification.

The invention additionally provides a method of determining an epitope in a sample. The method includes the steps of contacting a composition comprising a diverse population of reagent ligands attached to a solid support and a diverse population of antibodies specifically bound to the reagent ligands with a sample; and detecting the antibodies bound to the diverse population of reagent ligands. The method can further include the step of identifying which of the reagent ligands is unbound by antibody. In the method, a reagent ligand unbound by reagent antibody has binding activity for an antibody having specificity for a molecule in the sample.

The compositions of the invention for determining an epitope using antibodies or binding activity using binding molecules contain a diverse population of reagent ligands attached to a solid support. The reagent ligands are bound by a diverse population of reagent antibodies or reagent binding molecules. If desired, each of the ligands can be bound by antibody or binding molecules. This can be accomplished by removing any ligands from the solid support for which a corresponding binding antibody or binding molecule is not found. Alternatively, prior to addition of the sample, less than all of the reagent ligands can have bound molecules, for example, to use as a control or because corresponding binding molecules are not found. In such a case, the ligands having unbound antibodies or binding molecules can be tested prior to addition of sample and discarded or used as a control, as desired.

Thus, the invention provides a solid support comprising a diverse population of reagent ligands and a diverse population of reagent antibodies or reagent binding molecules specifically bound to the ligands, where all of the ligands are bound, about 99% of the ligands are bound, about 98% of the ligands are bound, about 95% of the ligands are bound about 90% of the ligands are bound, about 85% of the ligands are bound, about 80% of the ligands are bound, about 75% of the ligands are bound, about 70% of the ligands are bound, about 60% of the ligands are bound, about 50% of the ligands are bound, about 40% of the ligands are bound, about 30% of the ligands are bound, about 20% of the ligands are bound, about 10% of the ligands are bound, about 5% of the ligands are bound, or even less, if desired.

Proteins are formed by a series of amino acids linked together in long chains which fold into a 3-dimensional structure. Exposed on the surface of this structure are short peptide segments that are recognizable to antibodies. These antigenic peptides are called epitopes. Other epitopes include any antigenic determinant that can specifically bind to an antibody. By analogy to the terms genome and proteome, the epitome would be the entire collection of antigenic epitopes present in an organism.

The epitome is unique to an organism, a disease, or an individual. A map of the epitome would therefore provide convenient, quantitative information useful for identifying changes in the protein levels of diseased tissues and identifying different organisms by mapping all the antigenic surface peptides of the proteome. The epitome would also contain small molecule components and other antigenic biomolecules like oligosaccharides and oligonucleotides.

A diverse population of peptide ligands can be generated by methods well known to those skilled in the art. For example, the peptides can be synthesized by well known combinatorial methods (see, for example, Eichler et al., Med. Res. Rev. 15:481-496 (1995); Wilson and Czarnik, eds., Combinatorial Chemistry: Synthesis and Application, John Wiley & Sons, New York (1997); U.S. Pat. Nos. 5,264,563 and 5,405,783; Haridason et al., Proc. Indian Natl. Sci. Acad. Part A′ 53:717-728 (1987); Furka et al., Int. J. Peptide Protein Res. 37:487-493 (1991)). Methods of synthesizing nucleic acids or oligonucleotides ligands, oligosaccharide ligands, and organic molecule ligands are well known to those skilled in the art (see, for example, Ausubel et al., Current Protocols in Molecular Biology (Supplement 47), John Wiley & Sons, New York (1999); Sofia, Mol. Divers. 3:75-94 (1998); Eichler et al., Med. Res. Rev. 15:481-496 (1995); Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997)).

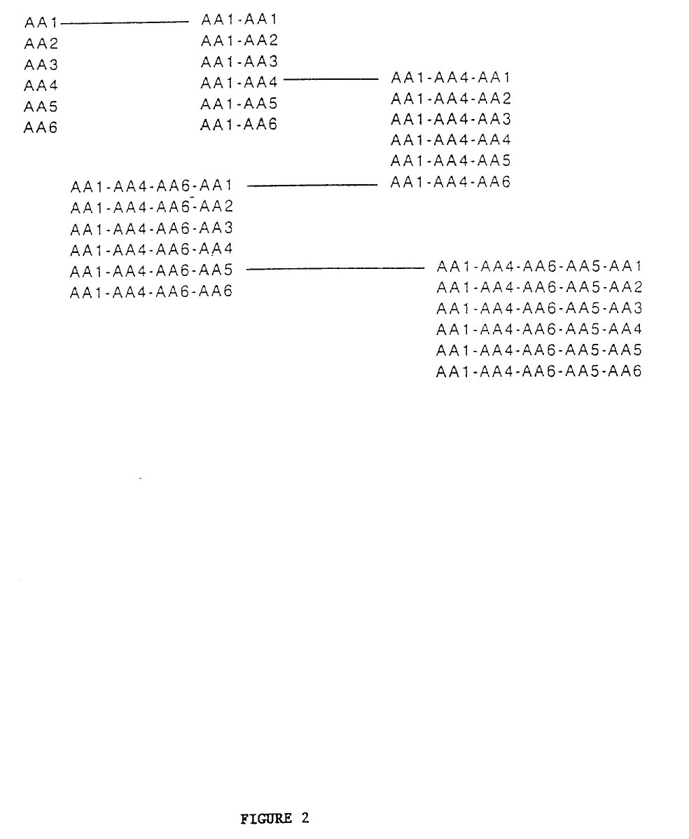

The epitome can be approximated in a combinatorial fashion by synthetically building ligand libraries, for example, peptide libraries, on a solid support in such a way that the peptide sequence is known based on its location on a ProtoChip. For example, a 5-mer peptide synthesized from 6 amino acids would result in 6⁵ (7776) possible combinations. A peptide library 5 amino acids long synthesized from the 20 naturally occurring amino acids would contain 20⁵ possible combinations, the equivalent to 3.2 million epitopes (FIG. 2). Thus, a diverse population of peptides can be synthesized that is representative of a large number of epitopes in a sample. Peptides of various lengths can be used, for example, 3-mer, 4-mer, 5-mer, 6-mer, 7-mer, 8-mer, 9-mer, 10-mer, 11-mer, 12-mer, 13-mer, 14-mer, 15-mer, 16-mer, 18-mer, 20-mer or longer peptides, or any convenient size of peptide so long as the peptide is capable of binding to an antibody.

The ligands can be displayed in microwell plates. Various microwell formats are commercially available including 2-well, 6-well, 12-well, 24-well, 96-well, 384-well, and 1536-well formats. A variety of materials have been used for the construction of microwell plates, including glass, polystyrene, polypropylene, polycarbonate, acrylonitrile butadiene styrene (ABS), and other plastics. In addition, a variety of coated microwell plates are commercially available that allow attachment of ligands to the surface through covalent bonds, electrostatic or hydrophobic interactions, or absorption.

Furthermore, the peptides can be conveniently displayed on an array. The spatially directed synthesis of peptides on an array has been demonstrated using photolabile protecting groups and photolithographic techniques developed in the microchip industry (Fodor, et al. Science, 251:767-773 (1991)).

The methods of the invention use a diverse population of ligands bound to a solid support. However, if desired, a method of identifying an epitope present in a sample can also be performed with a single ligand bound to a single antibody that can be displaced by an epitope in a sample.

A diverse population of antibodies can also be synthesized by methods well known to those skilled in the art, as described above (see, for example, Huse et al., Science 246:1275-1281 (1989)). Variability in antibody recognition is afforded by six complementarity-determining regions (CDRs) on the heavy and light chains of the antibody. By synthesizing the cDNA stretches that encode the complementarity-determining regions in a mixed pool random fashion and presenting them on various mouse antibody framework regions, a soluble antibody library can be prepared containing at least 10¹⁶ different antibodies (Breitling and Dubel, Recombinant Antibodies John Wiley, New York (1998)). This antibody library would present sufficient diversity to provide specific tight-binding antibodies for each of the combinatorial peptide epitope analogs or other ligand epitope analogs. Depending on the nature and complexity of the sample to be analyzed, the antibody library can be a naturally occurring library of antibodies expressed in an organism, in particular a mammal such as a human, primate, mouse, rabbit, goat, and the like, as disclosed herein (see Example I).

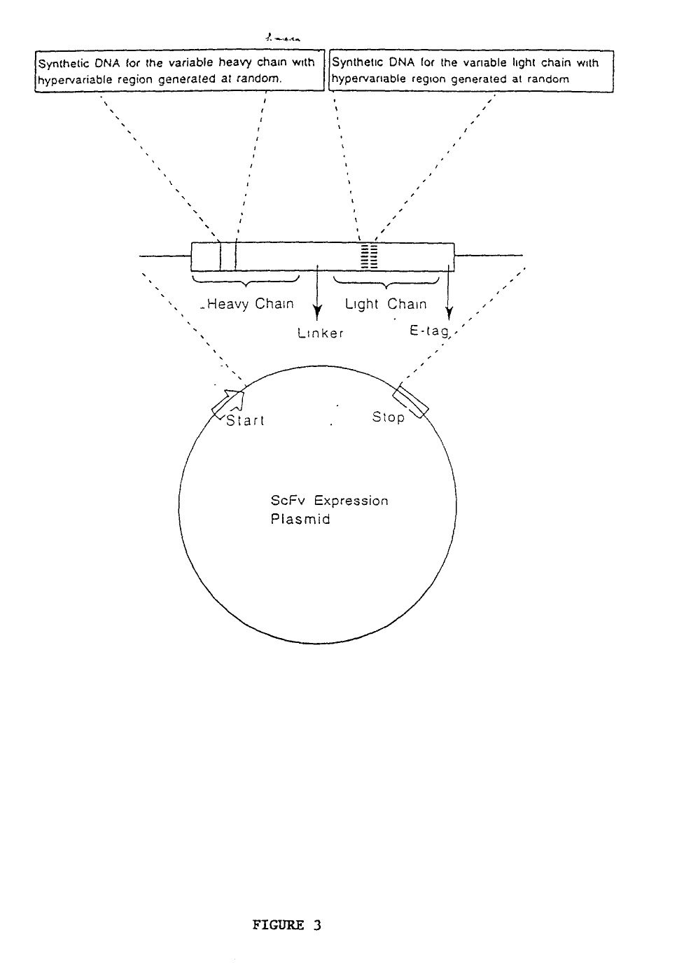

The form of the antibody used in the invention can be any of the well known forms described herein. A particularly useful form can be the ScFv form. The ScFv form of an antibody can be conveniently generated as a diverse population of antibodies for use in the invention (FIG. 3). The hypervariable regions in the heavy and light chain variable regions can be synthesized with a random DNA library that generates a diverse population of ScFv antibodies. Such an antibody library will have diverse binding affinities and specificities that can bind to the diverse population of ligands. Commercial systems are available for the expression of a recombinant antibody library (see, for example, Amersham Pharmacia Biotech; Piscataway N.J.).

Panning the antibody library over a high density peptide library such as a peptide chip, ligands immobilized on microwell plates, or other ligand libraries, allows the antibodies with the highest affinity to associate with a specific peptide or other ligand to generate the invention ProtoChip. Non-binding antibodies are removed by washing. Challenging the antibody bound ligands with a sample biological extract causes competing sample molecules that contain the same epitopes as the immobilized ligands to displace the antibody from the surface of the chip. Following washing, the remaining associated antibodies can be visualized using a variety of methods, as disclosed herein. For a 5 amino acid peptide library, the generated library would amount to 3.2 million individual, simultaneous immunoassays. As such, each of the epitopes would be both identified and quantified. The epitopes present generate a map of the protein extract.

The antibodies remaining bound to the diverse population of ligands attached to the solid support can be detected using well known methods. For example, an antibody can be directly modified or a secondary agent can be generated or modified to include a detectable moiety, for example, a radiolabel, a fluorochrome, a chromogen, a ferromagnetic substance, a luminescent tag, a detectable binding agent such as biotin, an enzyme such as horse radish peroxidase (HRP), alkaline phosphatase, glucose oxidase, and the like, or other detectable moieties known in the art that are detectable by analytical methods. A particularly useful detectable label is a fluorescent label. Methods suitable for detecting such moieties include, for example, fluorescence spectroscopy, autoradiography or phosphorimaging, calorimetric detection, light detection, or surface plasmon resonance.

As used herein, a label refers to single atoms and molecules that are either directly or indirectly involved in the production of a detectable signal. Any label can be linked to an antibody or secondary agent. These detectable atoms or molecules can be used alone or in conjunction with additional reagents. Such additional reagents are well-known in clinical diagnostic chemistry. The linking of a label to an antibody or secondary agent is well known in the art. Antibodies can be labeled by conjugating detectable labels, including enzymes, using cross linking agents or, if the antibodies are expressed recombinantly, for example, using antibody libraries, the antibodies can be labeled by expressing the antibodies as a fusion with a detectable peptide tag, for example, the E tag or similar peptide tags (see FIG. 3).

A secondary agent, which can specifically bind to an antibody, can also be directly labeled or be detectable by another reagent that is detectable. Thus, an antibody directly labeled or bound to a secondary agent that is labeled or detectable by another reagent can be detected using well known immunological detection methods (Harlow and Lane, supra, 1988; Harlow and Lane, Using Antibodies: A Laboratory Manual, Cold Spring Harbor Press (1999)).

The use of detectable labels is also convenient for quantitating the amount of epitope in a sample. A particularly useful detectable label for quantitation of the amount of epitope is a fluorescent label. Quantitative determinations can be made using well known methods for describing binding interactions. The relative concentration of an epitope can be related to fluorescence intensity. Specific epitopes can be quantified using a standard solution of the purified epitope and generating a calibration curve. Alternatively, the relative concentration for an unknown epitope can be determined in relation to its dissociation constant.

Although the methods of the invention are most conveniently used with a detectable label of either the antibodies or secondary agent, the binding of antibody can also be detected using mass spectroscopy, for example, matrix-assisted laser desorption-time of flight (MALDI-TOF) mass spectroscopy, if desired. Detection by MALDI-TOF analysis can also be used to determine partial sequences of the antibodies, for example, by determining the sequence of variable regions or CDRs of the detected antibodies.

The reagent ligands of the invention ProtoChip are conveniently attached to a solid support. The solid support can be a membrane such as a nylon or nitrocellulose membrane, glass, derivatized glass, silicon, plastic or other substrates. The ligands can be bound to a flat surface such as a membrane or plate or can be bound to spheres or beads. In one embodiment, the solid support can be in the form of a compact disc (CD).

A convenient format for the ligands can be an array containing a plurality of ligands. As used herein, an array refers to a format for presenting ligands where the ligands are stably bound to a solid support and arranged such that the binding to an antibody on the array can be detected. An array format is particularly convenient when the diverse population of ligands is a large population and is useful as a high density screening format.

For example, the format of the ProtoChip can take the form of a CD in which the ligand library is synthesized in discrete locations on the surface of the CD. In addition to encoded data, instructions and protocols using standard CD formatting, the ligand library such as a peptide library can be synthesized along the CD groove in discrete micron sized pits. The standard sized CD contains sufficient space to conservatively encode 310 million different peptides.

Audio CDs measure the reflection of an infrared photodiode laser's light from the surface of the CD. By decreasing the wavelength to 340 nm using commercially available photodiode laser and measuring the emitted light from fluorescently tagged antibodies or secondary agents, a table top confocal flourimeter can be constructed. Increased sensitivity arises from having the fluorophore immobilized on a solid support, which effectively reduces the sample volume to a range that would allow single molecule detection (Lu et al., Science, 282:1877-1882 (1998)). If desired, the methods of the invention using ProtoChip technology can be conveniently automated. Thus, coupled with a CD processing unit, the ProtoChip of the invention can be conveniently read using a desktop instrument in a doctors office or diagnostic laboratory. The ligands can also be attached in a multiwell format, if desired.

The ligands can be stably bound to a solid support via covalent interactions or non-covalent interactions so long as the ligands remain bound to the solid support during incubation or wash steps required for binding of antibodies and/or contacting with a sample. Generally, ligands are attached to a solid support, for example, through covalent bonds such as chemical crosslinks. A ligand can also be modified with an affinity tag that facilitates binding and or crosslinking of the ligand to the solid support.

The sample is contacted with the ProtoChip under conditions that allow specific binding of the sample molecules to the antibodies such that the antibodies are displaced from the ProtoChip. As used herein, specific binding means binding that is measurably different from a non-specific interaction. Specific binding can be measured, for example, by determining binding of a molecule compared to binding of a control molecule, which generally is a molecule of similar structure that does not have binding activity, for example, a peptide of similar size that lacks binding activity. Specificity of binding also can be determined, for example, by competition with a control molecule, for example, competition with an excess of the same molecule. In this case, specific binding is indicated if the binding of a molecule is competitively inhibited by itself. Thus, specific binding between an antibody and antigen is measurably different from a non-specific interaction and occurs via the antigen binding site of the antibody. An antigen such as a peptide has binding activity for the antibody if the antibody specifically binds to the peptide.

As used herein, selective binding refers to a binding interaction that is both specific and discriminating between molecules, for example, an antibody that binds to a single molecule or closely related molecules. For example, an antibody can exhibit specificity for an antigen that can be both specific and selective for the antigen if the epitope is unique to a molecule. Thus, a molecule having selective binding can differentiate between molecules, as exemplified by an antibody having specificity for an epitope unique to one molecule or closely related molecules. Alternatively, an antibody can have specificity for an epitope that is common to many molecules, for example, a carbohydrate that is expressed on a number of molecules. Such an antibody has specific binding but is not selective for one molecule or closely related molecules.

As used herein, the term “sample” is intended to mean any biological fluid, body fluid, cell, tissue, organ or portion thereof, that includes one or more different molecules that can function as antigens for antibodies bound to ligands on the ProtoChip or for binding molecules bound to ligands on the ProtoChip. The molecules in the sample are potential analyte molecules. The term includes samples obtained or derived from the individual. For example, a sample can be a fluid sample such as body fluid, including blood, plasma, urine, saliva or sputum. A sample can also be a tissue section obtained by biopsy, cells that are placed in or adapted to tissue culture, or fractions or components purified or extracted from a biological fluid, tissue or cell. When using a cell or tissue sample, the sample can be processed to generate an extract that can be conveniently contacted with a ProtoChip using methods well known to those skilled in the art (Harlow and Lane, supra, 1988; Harlow and Lane, supra, 1999).

If desired, the sample can be prepared with denaturants, including detergents such as sodium dodecyl sulfate (SDS). In the absence of denaturants, the epitopes accessible for binding to antibodies are the epitopes expressed on the surface of molecules, for example, the surface peptides of a folded protein. In the presence of denaturants, essentially all of the epitopes can become accessible, for example, due to unfolding of a protein and exposure of buried amino acid residues. Thus, conditions for treating the sample can be chosen to determine either epitopes accessible to antibody binding under native conditions or epitopes accessible under denaturing conditions.

The identity of the proteins or other molecules associated with increases or decreases in a given epitope can be obtained by comparing the epitope sequence to a sequence database such as that being generated by the human genome project. Alternatively, the protein of interest can be isolated using immunoaffinity techniques with the antibody specific for that epitope and sequenced using standard biochemical techniques. Mass spectroscopy can also be used to identify the antibody. In addition, the corresponding gene can be amplified from a cDNA library by polymerase chain reaction (PCR) using a degenerate primer corresponding to the epitope peptide. Methods of amplifying sequences by PCR are well known to those skilled in the art (Dieffenbach and Dveksler, PCR Primer: A Laboratory Manual, Cold Spring Harbor Press (1995); Ausubel et al., Current Protocols in Molecular Biology (Supplement 47), John Wiley & Sons, New York (1999))

The invention further provides a method of diagnosing a disease. The method includes the steps of contacting a composition comprising a diverse population of reagent ligands attached to a solid support and a diverse population of reagent antibodies specifically bound to the reagent ligands with a sample from an individual; detecting the reagent antibodies bound to the diverse population of reagent ligands; and identifying which of the reagent ligands is unbound by reagent antibody, wherein a reagent ligand unbound by reagent antibody has binding activity for an antibody having specificity for a molecule associated with the disease.

The methods of the invention can be applied to generate a database of epitope maps for a variety of tissues, causative proteins or those affected by a disease, which can be readily identified and quantified. Since the methods of the invention are used to measure epitopes as opposed to whole protein sequences, changes in post translational modification and proteolytic processing can also be directly identified. The methods of the invention can be used to determine if an individual has a particular disease such as cancer, Alzheimer's disease, cardiovascular diseases, cerebrovascular diseases, congenital anomalies, infectious diseases, parasitic diseases, endocrine related diseases, nutritional diseases, metabolic diseases, metabolic disorders, diabetes, blood diseases, mental disorders, diseases of the nervous system, circulatory diseases, respiratory diseases, digestive diseases, genitourinary diseases, skin diseases, perinatal conditions, inflammatory diseases, arthritis, erectile or fertility disorders, renal diseases, liver diseases, and gastrointestinal diseases.

The methods of the invention are therefore useful for diagnostic applications. The high specificity of antibodies make them invaluable diagnostic tools. To date, the development of antibody based diagnostics has required a prior knowledge of the antigen. The identification of these antigens in many cases is the result of years of academic and industrial research. Subsequently, specific epitopes on the antigen must be identified, analogs synthesized, and injected into mice in order to generate monoclonal antibodies which are frequently nonspecific or have poor binding characteristics. Since the present invention is directed to measuring the epitome, analysis of biological fluids can immediately generate a panel of specific tight binding antibodies for disease related proteins without requiring any prior knowledge of the antigen.

If desired, specific antibodies can be recreated by immunization of mice with the identified epitope to generate monoclonal antibodies. In addition, specific antibodies can be generated by analyzing biological fluids using a phage display antibody library or by panning an antibody library over the ligand followed by isolation and sequence analysis of the recombinant antibody. However, identification of specific antibodies is not required since a disease specific ProtoChip can be produced based on disease specific epitopes identified by methods of the invention. In another embodiment, a diagnostic ProtoChip can be produced that holds the epitopes that are diagnostic for a wide variety of diseases or medical conditions.

The method of the invention can be used to identify therapeutically useful antibodies. For example, the identification of a tumor specific epitope using a ProtoChip of the invention also provides the tumor specific antibody associated with that epitope. This antibody can useful therapeutically for the treatment of cancer. Examples of antibody therapeutics for the treatment of cancer include Herceptin and Rituxan. Due to the specificity of the identified antibodies for the tumor, the antibody can be used to target a tumor for therapeutic or diagnostic purposes, or other disease targets, as desired.

The methods of the invention can also be used to identify antigens useful in the development of vaccines. Screening an infectious agent using methods of the invention using, for example, a ProtoChip, allows identification of epitopes associated with the infectious agent. The epitope can be used for preparation of a vaccine, for example, by coupling the epitope to a suitable carrier, and administered to an individual in a pharmaceutical composition suitable for stimulating an immune response. Such compositions suitable for stimulating an immune response are well known to those skilled in the art and can include, for example, a physiologically acceptable carrier and/or an adjuvant suitable for stimulating an immune response, as desired.

The methods of the invention can be conveniently automated, if desired. Following automatic washing and reagent additions within a ProtoChip analyzer, the ProtoChip can be quantified, for example, using fluorescence to detect bound antibodies. By applying a droplet of body fluid on a diagnostic ProtoChip and placing the chip into a processor and reader, immediate in-office diagnostics can be applied to a panel of disorders. The generated epitope fingerprint is compared to a database of values that results in an easily interpreted readout of the diagnosis. Among the many foreseeable diagnostic applications, ProtoChips specific for vascular diseases, neurological disorders, metabolic diseases, or infectious diseases can be produced in addition to an all purpose panel useful for annual checkups.

An advantage of the present invention using ProtoChip based diagnostics is that panels of antibodies can be generated without any prior knowledge or prejudices of the disease. Additionally, with the appropriate fluids, specific diagnostics can be generated in a matter of days or weeks as opposed to the current standard of months or years. The present invention provides more specificity as a result of multiple epitope probes, more flexibility as a result of the ability to multiplex different diagnostics on the same chip, and, as a result of the ease of discovery, a shorter product development time than other immunoassay diagnostics.

The methods of the invention are also useful for drug development and pharmacoproteomic applications. The unachieved goal of genetically characterizing patient populations in order to more efficiently target drugs to those who would respond has been termed pharmacogenomics. Three markets have been suggested for this proposed application of genomic techniques: 1) assisting in drug development at the clinical trial stage by targeting patient populations who will most benefit, 2) reanalysis of approved drugs that show disappointing efficacy in order to reposition the patient population to those who are most likely to improve, 3) reviving failed drug candidates by weeding out patients prone to side effects or non-response. As yet, pharmacogenomics has not become a reality in part due to the poor correlation between mRNA levels and biological response. Unlike genomic approaches, the present invention allows for the quantification of protein levels. As such, clinical trials have a greater chance of success if the epitome of the patient population is mapped to a homogenous group of responders, the efficacy of marketed drugs can be optimized to prescription practice as they change resulting from analysis using the invention ProtoChip, and failed drugs can be revived as the result of uncovering the patient requirements through ProtoChip mapping. The goals set forth for pharmacogenomics can be realized using invention ProtoChip technology by analyzing the epitome of the patient population.

The methods of the invention thus can be used to provide information useful in drug development. For example, if insulin were to be tested against a random population of diabetics, it would likely show no significant effect on the lowering of glucose levels. It is only after selecting a group of subjects based upon age of onset of symptoms that the therapeutic value of insulin is realized for juvenile onset diabetes. In the design of clinical trials, the selection of the wrong patient subpopulation for the study or the lack of selection criteria can lead to the failure of a potentially valuable drug. By prescreening trial candidates using methods of the invention, a near homogeneous group of patients can be enlisted in order to ensure the greatest chances for success.

Alternatively, ProtoChip analysis of patients from an unbiased trial population can uncover specific markers suggestive of the potential outcome of treatment. Accordingly, without stratifying the patients prior to the trial, it is possible that those subjects with a given amount of a specific epitope show a greater chance for responding to the drug. This observation can be taken forward to the design of epitome based parameters for the prescription of drugs. While this strategy can serve to reduce the patient population to only those who respond to a drug, the improved accuracy of prescriptions can generate new markets for drugs that previously showed limited efficacy or by reviving drugs that failed to prove sufficient efficacy during clinical trials. Thus, methods of the invention can be used in new clinical trials for drugs that failed to show statistically significant efficacy in previous clinical trials.

The methods of the invention can be used to determine the epitome map and generate databases describing the epitome for a variety of organisms. These databases can include various pathogenic species, healthy and diseased tissues from humans and economically valuable animal species, drug efficacy profiles, plants, insects, and other organisms such as bacteria, yeasts and immortalized cell lines. Thus, the methods of the invention are useful for identifying a species of organism such as a species animal, plant or bacteria.

For example, a particular bacterium or strain of bacterium can be identified using methods of the invention. The methods can be used to identify various bacteria such as pathogenic bacteria. For example, a pathogenic strain such as a methacillin-resistant Staphylococcus aureus strain can be identified using methods of the invention. The precise identification of a bacterial strain in a sample can be used to select an appropriate antibiotic effective against the particular organism.

Specific proteins can be mapped using protein standards or proteins purified using the identified antibody can be sequenced such that changes in the epitome are correlated to a specific protein or group of proteins. The databases identified by methods of the invention are useful for the discovery of new pharmacological targets, new agricultural traits, insecticides and the development of diagnostic tools. The methods of the invention can be used in diagnostic applications such that a physician can place biological samples into a ProtoChip reader and immediately be provided with the identity of infectious bacteria or viruses and the recommended treatment guidelines based upon that specific organism and its resistance profile.

The invention can also be used without a combinatorial antibody library bound to the ligands. Instead, a protein of interest can be applied to the immobilized ligands. Evaluation of bound protein can be used to identify ligands for the protein. These ligands can then be used as leads for drug optimization, target validation tools for pharmacology models, or for the development of high throughput screening assays. This method eliminates the need for any prior knowledge of protein function or activity and allows a single assay protocol to be used for high throughput screens.

The invention further provides a method of mapping accessible epitopes of a polypeptide. The method includes the steps of contacting a composition comprising a diverse population of reagent ligands attached to a solid support and a diverse population of reagent antibodies specifically bound to the reagent ligands with a polypeptide; detecting the reagent antibodies bound to the diverse population of reagent ligands; and identifying which of the reagent ligands is unbound by reagent antibody, wherein a reagent ligand unbound by reagent antibody has binding activity for an antibody having specificity for a polypeptide epitope accessible to the antibody. Such a method is particularly useful when the ligands are peptides.

The methods of the invention can also be used for protein structural determinations. The value of genome sequence information is only realized upon determination of the functional significance of the encoded proteins. This function is imparted not through the primary structure of the sequence itself but through the tertiary structure, the three dimensional shape of the protein. Structure determination methods have had limited success in accurately predicting the structure of a protein based solely on its sequence. The experimental determination of a protein structure is slow and tedious. Since the epitope map identifies surface peptides of a protein, the methods of the invention using a specific protein in place of the biological fluid sample provide experimental structural information that can be coupled with sequence information to predict the tertiary protein structure. These predictions can be refined by structure or sequence comparison to proteins with known structure and function. The methods of the invention can thus be used for the rapid functional analysis of genomic and proteomic leads without the need to express and isolate large amounts of protein and without the investment of large amounts of time as is required using traditional structural methods.

The identification of surface epitopes can be combined with computational protein structure prediction algorithms, including ab initio folding algorithms such as the strings method (Moult, Curr. Opion. Biotechnol. 10:583-588 (1999); Selbig et al., Bioinformatics 15:1039-1046 (1999); Osguthorpe, Curr. Opin. Struct. Biol. 10:146-152 (2000); Jonassen et al., Proteins 34:206-219 (1999)). Computational protein structure algorithms are well known to those skilled in the art. The combination of the identification of surface epitopes and folding algorithms allows a more accurate prediction of tertiary protein structure than with computational methods alone. Competition with the ProtoChip and a purified protein allows identification of the surface epitopes of the protein. Under the constraint of having these epitopes on the surface of the protein, there are fewer degrees of freedom, for example, fewer low energy states, accessible to the computational calculation. Thus, the combination of the methods of the invention directed to identifying surface epitopes of a protein with computational protein structure prediction algorithms can be used to greatly improve the accuracy and structure prediction of polypeptides.

The determination of the three dimensional structure of a protein has become a key component of drug discovery. Currently this is accomplished through X-ray crystallography or by NMR. Both of these methods are limited by the physical properties of the protein, its solubility, and its ability to crystallize. Frequently, the determination of the three dimensional structure takes a year or more. With the identification of hundreds of potential targets from genomic and proteomic studies, a method to calculate the three dimensional structure based upon the protein sequence would accelerate the drug discovery process. The epitope map generated using the invention ProtoChip for a given protein provides a low resolution map of the protein that, when used in conjunction with computational methods, can yield accurate representations of the protein.

Immunoaffinity purification of proteins is hampered by the difficulty in identifying appropriate antibodies for the protein of interest. The protein must first be purified in sufficient quantities to immunize rabbits for the production of polyclonal antibodies or mice for monoclonal antibodies. If a satisfactory immune response is obtained, then the antibodies can be immobilized on a solid support to make an immunoaffinity column. As a result of the high affinity of traditionally prepared monoclonal or polyclonal antibodies, elution of the studied protein from an immunoaffinity column frequently results in the denaturation of the protein. Therefore, the antibodies raised against the protein are often not satisfactory for use in purification columns. Using the ProtoChip of the present invention, an antibody for any protein, without prior purification or even characterization of that protein, can be generated having a predefined dissociation constant selected for binding characteristics based on wash conditions in the ProtoChip analysis. Exemplary variable wash conditions include changing the pH, changing ionic strength, changing temperature, changing wash time, or any combination thereof. For example, using higher stringency wash conditions such as increasing ionic strength and/or varying other buffer components and conditions can be used to select for antibodies having tighter binding activity for the ligand. Therefore, the invention ProtoChip can be used to develop specific immunoaffinity columns for any protein.

While the invention ProtoChip and related methods are useful in human health applications, the methods of the invention can similarly be applied to animal health and agricultural uses. The epitope map can be determined using methods of the invention and used in quality control, for example, of meat processing, animal breeding programs, and disease screening. The ability to quickly establish specific epitope maps can be used to boost the success of captive breeding programs by maximizing phenotypic rather than genotypic diversity.

Agricultural applications of the invention methods can be extended to plants with the characterization and identification of proteins that impart beneficial effects such as insect resistance or improved growth characteristics of a crop plant. Plant epitome characterization can also be used in the identification and classification of different plants. Plant characterization can be useful in the development of novel pharmaceuticals. For example, taxol was discovered in the bark of the rare slow growing Taxus brevifolia. Due to the scarcity of this plant, production of this valuable drug was economically limited. Tedious analysis of other plants in the Taxus family showed that the common fast growing Taxus baccata produced a chemically similar compound in its leaves that is easily converted to the biologically active drug taxol. Other examples of plant-derived drugs include the lymphoma drug vinblastine, which is derived from the Madagascar rosy periwinkle, and the muscle relaxant curare, which is derived from the South American curare vine. Similar botanical findings using methods of the invention can prove useful in drug discovery while preserving ecologically susceptible species.

The methods of the invention can also be used to identify drug targets and are therefore useful in drug discovery. Comparison of the epitope map of a biological fluid from a healthy individual to the epitope map of a biological fluid from a diseased individual can be used to reveal epitopes specific for the disease state. By identifying the protein associated with these disease-associated epitopes, potential therapeutic targets can be determined.

The invention additionally provides a method of identifying a potential therapeutic target. The method includes the steps of contacting a composition comprising a diverse population of reagent ligands attached to a solid support and a diverse population of reagent antibodies specifically bound to the reagent ligands with a sample from an individual having a disease; detecting reagent antibody binding to the diverse population of reagent ligands; comparing the reagent antibody binding to the diverse population of reagent ligands to the reagent antibody binding of a normal sample contacted with the composition; and determining which of the reagent ligands differs in reagent antibody binding between the sample from the individual having a disease and the normal sample, wherein a reagent ligand differing in reagent antibody binding between the samples is a potential therapeutic target.

Comparison of antibody binding in a sample from a diseased individual to a normal sample, that is, a sample from an individual not having the disease, can be used to determine epitopes related to the disease based on differences in antibody binding. A ligand of the invention composition that differs in binding between these samples is a potential therapeutic target. If desired, a group of diseased individuals can be analyzed and compared to a group of normal individuals, that is, individuals not having the disease. A statistically significant number of individuals can be selected for the groups and used for comparison to determine which ligands differ in antibody binding. For example, 50 individuals can be selected for a group. The ligands that differ in antibody binding between the samples can be further characterized by the methods disclosed herein and used as a potential therapeutic target to screen for drug candidates useful in treating the disease.

The compositions and methods disclosed above use antibodies bound to ligands. However, it is understood that other binding molecules can be used to bind to ligands for detecting the presence of a corresponding binding activity in a sample using the methods disclosed herein using antibodies. Other binding molecules can include polypeptides, receptors, enzymes, carbohydrates, lipids, and the like, so long as the binding molecule can bind to the reagent ligand and has the ability to potentially bind to a corresponding sample molecule, such that displacement of the binding molecule can be used to detect the presence of a molecule in the sample, as disclosed herein.

When using antibodies attached to ligands, the binding activity in the sample identified by methods of the invention is referred to as an epitope. In the case of using binding molecules other than antibodies, the binding activity of the sample molecules is determined. Accordingly, a sample that displaces a binding molecule from a ligand has a binding activity for that binding molecule, analogous to an epitope when an antibody is used.

Thus, the invention provides a method of determining a binding activity in a sample. The method includes the steps of contacting a composition comprising a diverse population of reagent ligands attached to a solid support and a diverse population of reagent binding molecules specifically bound to the reagent ligands with a sample; and detecting the reagent binding molecules bound to the diverse population of reagent ligands. The method can further comprise the step of identifying which of the reagent ligands is unbound by reagent binding molecule. The reagent ligand unbound by reagent molecule has binding activity for a binding molecule having specificity for a molecule in the sample.

It is understood that modifications which do not substantially affect the activity of the various embodiments of this invention are also included within the definition of the invention provided herein. Accordingly, the following examples are intended to illustrate but not limit the present invention.

### EXAMPLE I

### Epitope Mapping of Plasmodium falciparum Merozoite Surface Protein 1

This example describes mapping of epitopes of the 19 kDa C-terminal region of merozoite surface protein 1 (MSP1-19) from Plasmodium falciparum.

A natural human IgG antibody library was tested for its ability to bind to peptides associated with the 19 kDa C-terminal region of merozoite surface protein 1 (MSP1-19) from Plasmodium falciparum (Kaslow et al., Mol. Biochem. Parasitology 63:283-289 (1994)). The 89 amino acid sequence from MSP1-19 was used for the epitope mapping experiment (see Table 1).

Briefly, a library of pentamer peptides was synthesized on polypropylene pins following the procedures described by Geysen et al., Proc. Natl. Acad. Sci. USA 81:3998-4002 (1984). These peptides represented all five-amino-acid stretches of MSP1-19 offset by one residue (Table 1). Peptide pins were precoated in phosphate buffered saline (PBS), pH 7.2, containing 2% BSA and 0.1% TWEEN 20 for one hour at room temperature. Five successive washes of the pins were carried out for five minutes each with agitation in PBS. Human IgG (Calbiochem; San Diego Calif.) was complexed to the peptides by incubating 0.1 mg/mL IgG in PBS at 4° C. for 30 hours. Unbound antibody was removed by washing as described above in 10 mM TRIS, pH 7.4 buffer containing 150 mM NaCl (TBS) using new microtiter plates for transferring the pins for each of the five washings. Anti-human IgG (goat) alkaline phosphatase conjugate was diluted to 0.1 mg specific antibody/mL in TBS and incubated with the peptide/antibody complex for one hour at room temperature before washing five times with TBS, as described above. The pins were then incubated in assay solution containing 10 mM TRIS, pH 8.0, 150 mM NaCl, 0.5 MM MgCl₂, and 0.1 mM 4-methylumbelliferyl-phosphate for 30 minutes at room temperature in the dark. Following incubation, the fluorescence intensity of the assay solution was measured in a Spectromax Gemini plate reader (Molecular Devices; Sunnyvale Calif.)(ex 358 nm/em 450 nm).

The peptide/antibody complexes on the pins were washed five times in TBS, as described above, and incubated with 50 mg/mL MSP1-19 diluted in TBS for one hour at room temperature. The pins were washed in TBS as before and incubated in assay mixture for 30 minutes in the dark prior to measurement of the fluorescent intensity. Change in binding was determined using the equation:

ΔF=(F_{MSP}−F_{b,MSP})/(F_{100,MSP}−F_{b,MSP})−(F₀^{-F}_{b,0})/(F^{100,0}−F_{b,0})

where F_{MSP} and F₀ are the fluorescence intensities of peptide containing pins after and before exposure to MSP1-19, respectively. F_{b,MSP} and F_{b,0} are the fluorescence intensities of pins with no peptide after and before exposure to MSP1-19, respectively. F_{100,MSP} and F_{100,0} are the fluorescence intensities of pins containing a control peptide, GLAQG (SEQ ID NO:90), after and prior to exposure to MSP1-19.

Human IgG complexed with all peptide-containing pins, with an average relative fluorescent intensity of 46744 (arbitrary units) while control pins without peptides had an average relative fluorescence of 244. The large fluorescence relative to the blank indicates human IgG bound to the peptides, while non-specific binding was not observed to pins lacking bound peptides. The pins were pre-exposed to BSA. If significant amounts of BSA were to bind to the pins, it would be expected that the IgG would associate with BSA on the surface of the pins as a result of IgG affinity for BSA. The absence of IgG on the control pins indicates that BSA does not associate in a non-specific fashion with the pins under the assay conditions. The range of relative fluorescence for the peptide pins was 25560 to 57880, suggesting a gradient of binding affinities and population density of the specific peptide-binding antibodies. Exposure of the antibody/peptide pins to MSP1-19 caused a decrease in fluorescence of greater than 10% in eleven peptides associated with two regions corresponding to the sequences C49-D57 and N70-D88 (Table 1). There was no significant decrease in fluorescence of control peptides upon exposure to MSP1-19. Therefore, the antibodies bound to the peptides dissociated from the pins as the result of competition by equivalent epitopes on MSP1-19.

TABLE 1
Epitope Map of MSP1-19

| Pep- | Sequ- | ΔF | Pep- | Sequ- | ΔF | Pep- | Sequ- | ΔF |
|---|---|---|---|---|---|---|---|---|
| tide # | ence | (%) | tide # | ence | (%) | tide # | ence | (%) |
| 1 | NISQH | <5 | 31 | LLNYK | <5 | 61 | KCTEE | 6 |
| 2 | ISQHQ | <5 | 32 | LNYKQ | <5 | 62 | CTEED | <5 |
| 3 | SQHQC | <5 | 33 | NYKQE | <5 | 63 | TEEDS | <5 |
| 4 | QHQCV | <5 | 34 | YKQEG | <5 | 64 | EEDSG | 7 |
| 5 | HQCVK | <5 | 35 | KQEGD | <5 | 65 | EDSGS | 7 |
| 6 | QCVKK | <5 | 36 | QEGDK | <5 | 66 | DSGSN | 9 |
| 7 | CVKKQ | <5 | 37 | EGDKC | <5 | 67 | SGSNG | <5 |
| 8 | VKKQC | <5 | 38 | GDKCV | <5 | 68 | GSNGK | <5 |
| 9 | KKQCP | <5 | 39 | DKCVE | <5 | 69 | SNGKK | <5 |
| 10 | KQCPQ | <5 | 40 | KCVEN | <5 | 70 | NGKKI | 12 |
| 11 | QCPQN | <5 | 41 | CVENP | <5 | 71 | GKKIT | 6 |
| 12 | CPQNS | <5 | 42 | VENPN | <5 | 72 | KKITC | 9 |
| 13 | PQNSG | <5 | 43 | ENPNP | <5 | 73 | KITCE | 7 |
| 14 | QNSGC | <5 | 44 | NPNPT | <5 | 74 | ITCEC | 5 |
| 15 | NSGCF | <5 | 45 | PNPTC | <5 | 75 | TCECT | 12 |
| 16 | SGCFR | <5 | 46 | NPTCN | <5 | 76 | CECTK | <5 |
| 17 | GCFRH | <5 | 47 | PTCNE | <5 | 77 | ECTKP | 13 |
| 18 | CFRHL | <5 | 48 | TCNEN | <5 | 78 | CTKPD | 10 |
| 19 | FRHLD | <5 | 49 | CNENN | 16 | 79 | TKPDS | 11 |
| 20 | RHLDE | <5 | 50 | NENNG | 5 | 80 | KPDSY | 11 |
| 21 | HLDER | <5 | 51 | ENNGG | <5 | 81 | PDSYP | 9 |
| 22 | LDERE | <5 | 52 | NNGGC | 6 | 82 | DSYPL | 9 |
| 23 | DEREE | <5 | 53 | NGGCD | 10 | 83 | SYPLF | 13 |
| 24 | EREEC | <5 | 54 | GGCDA | <5 | 84 | YPLFD | 12 |
| 25 | REECK | <5 | 55 | GCDAD | 7 | 85 | PLFDG | 16 |
| 26 | EECKC | <5 | 56 | CDADA | <5 | 86 | LFDGI | 11 |
| 27 | ECKCL | <5 | 57 | DADAK | <5 | 87 | FDGIF | 9 |
| 28 | CKCLL | <5 | 58 | ADAKC | <5 | 88 | DGIFC | 7 |
| 29 | KCLLN | <5 | 59 | DAKCT | <5 | 89 | GIFCS | 11 |
| 30 | CLLNY | <5 | 60 | AKCTE | 5 | | | |

Peptides 1-89 correspond to SEQ ID NOS:1-89, respectively.

The most commonly identified serum anitibody response for Kenyan malaria immune positive donors to MSP1-19 peptides corresponded to C78-G91 (Egan et al., Infection and Immunity 65:3024-3031 (1997)). This sequence overlaps with the N70-D88 epitope region identified by epitope mapping in this study. The study by Egan et al. showed that the region corresponding to the C49-D57 epitope was observed at a lower frequency as a serum antibody response, while other infrequently observed MSP1-19 epitopes were also identified.

The maximum amount of antibody dissociated from the peptide as the result of exposure to MSP1-19 was 16%. The x-ray structure of MSP1-19 shows that the majority of the amino acid residues in this protein are solvent exposed and would be expected to have the potential to bind antibodies. Epitope mapping, however identified only two regions with significant IgG binding affinity. C49-D57, corresponding to a short β-sheet on the protein surface, and N70-D88, a long strand of a β-sheet exposed to the surface, were identified as epitopes, while the inaccessible antiparallel strand was not identified as an epitope.

These results demonstrate that antibody/peptide arrays can be formed by the combination of peptide libraries and antibody libraries. Furthermore, these results demonstrate that antibodies from a peptide library will associate with an antibody library, and those antibodies can be dissociated upon exposure to a competing protein or peptide.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit of the invention.

I claim:

1. A composition comprising a diverse population of reagent ligands attached to a solid support and a diverse population of reagent antibodies specifically bound to said reagent ligands.

2. The composition of claim 1, wherein each of said reagent ligands is bound to a reagent antibody.

3. The composition of claim 1, wherein said reagent ligands are selected from the group consisting of peptides, oligosaccharides, oligonucleotides, and organic molecules.

4. The composition of claim 1, wherein said reagent ligands are on an array.

5. The composition of claim 1, wherein said reagent antibodies are labeled.

6. The composition of claim 5, wherein said label is a fluorescent label.

7. A composition comprising a diverse population of reagent ligands attached to a solid support and a diverse population of reagent antibodies specifically bound to a subset of said reagent ligands, wherein an unbound reagent ligand has binding activity for a reagent antibody having specificity for a molecule in a sample.

8. The composition of claim 7, wherein said reagent ligands are selected from the group consisting of peptides, oligosaccharides, oligonucleotides, and organic molecules.

9. The composition of claim 7, wherein said reagent ligands are on an array.

10. The composition of claim 7, wherein said reagent antibodies are labeled.

11. The composition of claim 10, wherein said label is a fluorescent label.

12. A method of determining an epitope in a sample, comprising:
(a) contacting a composition comprising a diverse population of reagent ligands attached to a solid support and a diverse population of reagent antibodies specifically bound to said reagent ligands with a sample; and
(b) detecting said reagent antibodies bound to said diverse population of reagent ligands.

13. The method of claim 12, further comprising the step of identifying which of said reagent ligands is unbound by reagent antibody.

14. The method of claim 12, wherein said reagent ligand unbound by reagent antibody has binding activity for an antibody having specificity for a molecule in said sample.

15. The method of claim 12, wherein said reagent ligands are selected from the group consisting of peptides, oligosaccharides, oligonucleotides, and organic molecules.

16. The method of claim 12, wherein said sample is selected from the group consisting of a cell, a tissue, a body fluid, and an organism.

17. The method of claim 12, wherein said tissue is a biopsy from an individual with a disease.

18. The method of claim 12, wherein said sample is a species of animal or plant.

19. The method of claim 12, wherein said reagent ligands are on an array.

20. The method of claim 12, wherein said reagent antibodies are labeled.

21. The method of claim 20, wherein said label is a fluorescent label.

22. A method of diagnosing a disease, comprising:
(a) contacting a composition comprising a diverse population of reagent ligands attached to a solid support and a diverse population of reagent antibodies specifically bound to said reagent ligands with a sample from an individual;
(b) detecting said reagent antibodies bound to said diverse population of reagent ligands; and
(c) identifying which of said reagent ligands is unbound by reagent antibody, wherein a reagent ligand unbound by reagent antibody has binding activity for an antibody having specificity for a molecule associated with said disease.

23. The method of claim 22, wherein said reagent ligands are selected from the group consisting of peptides, oligosaccharides, oligonucleotides, and organic molecules.

24. The method of claim 22, wherein said reagent ligands are on an array.

25. The method of claim 22, wherein said reagent antibodies are labeled.

26. The method of claim 25, wherein said label is a fluorescent label.

27. A method of identifying a potential therapeutic target, comprising:
(a) contacting a composition comprising a diverse population of reagent ligands attached to a solid support and a diverse population of reagent antibodies specifically bound to said reagent ligands with a sample from an individual having a disease;
(b) detecting reagent antibody binding to said diverse population of reagent ligands;
(c) comparing said reagent antibody binding to said diverse population of reagent ligands to the antibody binding of a normal sample contacted with said composition; and
(d) determining which of said reagent ligands differs in antibody binding between said sample from said individual having a disease and said normal sample, wherein a reagent ligand differing in antibody binding between said samples is a potential therapeutic target.

28. The method of claim 27, wherein said reagent ligands are selected from the group consisting of peptides, oligosaccharides, oligonucleotides, and organic molecules.

29. The method of claim 27, wherein said reagent ligands are on an array.

30. The method of claim 27, wherein said reagent antibodies are labeled.

31. The method of claim 30, wherein said label is a fluorescent label.

32. The method of claim 27, wherein the reagent antibody displaced from said reagent ligands differing in antibody binding is a potential therapeutic antibody.

33. A method of mapping accessible epitopes of a polypeptide, comprising:
(a) contacting a composition comprising a diverse population of reagent ligands attached to a solid support and a diverse population of reagent antibodies specifically bound to each of said reagent ligands with a polypeptide;
(b) detecting said reagent antibodies bound to said diverse population of reagent ligands; and
(c) identifying which of said reagent ligands is unbound by reagent antibody, wherein a reagent ligand unbound by reagent antibody has binding activity for an antibody having specificity for a polypeptide epitope accessible to said antibody.

34. The method of claim 33, wherein said reagent ligands are peptides.

35. The method of claim 33, wherein said reagent ligands are on an array.

36. The method of claim 33, wherein said reagent antibodies are labeled.

37. The method of claim 36, wherein said label is a fluorescent label.

38. A method of determining a binding activity in a sample, comprising:
(a) contacting a composition comprising a diverse population of reagent ligands attached to a solid support and a diverse population of reagent binding molecules specifically bound to said reagent ligands with a sample; and
(b) detecting said reagent binding molecules bound to said diverse population of reagent ligands.

39. The method of claim 38, further comprising the step of identifying which of said reagent ligands is unbound by reagent binding molecule.

40. The method of claim 38, wherein said reagent ligand unbound by reagent molecule has binding activity for a binding molecule having specificity for a molecule in said sample.

41. The method of claim 38, wherein said reagent ligands are selected from the group consisting of peptides, oligosaccharides, oligonucleotides, and organic molecules.

42. The method of claim 38, wherein said sample is selected from the group consisting of a cell, a tissue, a body fluid, and an organism.

43. The method of claim 38, wherein said tissue is a biopsy from an individual with a disease.

44. The method of claim 38, wherein said sample is a species of animal or plant.

45. The method of claim 38, wherein said reagent ligands are on an array.

46. The method of claim 38, wherein said reagent binding molecules are labeled.

47. The method of claim 38, wherein said label is a fluorescent label.
